(19) United States
(12) Patent Application Publication
(10) Pub. No.: US 2023/0015857 A1
(43) Pub. Date: Jan. 19, 2023

(54) METHOD TO REDUCE BOTH VOCS AND CO2 IN LIVING AND WORKING SPACES

(71) Applicant: TOP PRODUCT INNOVATIONS, INDIAN TRAIL, NC (US)

(72) Inventors: Robert E. Miller, INDIAN TRAIL, NC (US); John Newport, INDIAN TRAIL, NC (US); Alan Rosenberg, INDIAN TRAIL, NC (US)

(21) Appl. No.: 17/785,249

(22) PCT Filed: Jan. 21, 2021
(86) PCT No.: PCT/US2021/014370

§ 371 Date: Jun. 14, 2022

Related U.S. Application Data
(60) Provisional application No. 62/963,617, filed on Jan. 21, 2020.

(51) Int. Cl. B01D 53/14; A61L 9/22; F24F 8/30; B01D 53/00; B01D 53/06; B01D 53/18

(52) U.S. Cl. CPC B01D 53/1475; A61L 9/22; F24F 8/30; B01D 53/007; B01D 53/06; B01D 53/1425; B01D 53/18; A61L 2209/16; B01D 2257/504; B01D 2257/708; B01D 2252/103; B01D 2252/2023; B01D 2252/204; B01D 2252/30; B01D 2259/4508; B01D 2259/4009

(57) ABSTRACT

A system and method to both reduce interior levels of volatile organic compounds and carbon dioxide that is more energy efficient than increasing building ventilation rates. The system comprises a carbon dioxide scrubber that operates in a continuous manner, and optionally but preferably further comprises an air purifier.

### CROSS-REFERENCE TO RELATED APPLICATIONS

This application is a National Phase Application of International Patent App. No. PCT/US2021/014370, filed Jan. 21, 2021, which claims the benefit of U.S. Provisional Patent App. No. 62/963,617, filed Jan. 21, 2020, the entire disclosures of both of which are hereby incorporated by reference as if set forth in their entirety herein.

### BACKGROUND

Energy efficient buildings, such as residential living places and commercial building working spaces typically have a lower interior air turnover than better ventilated but typically less energy efficient buildings. This lower turnover of inside air with outside air allows the level of pollutants such as volatile organic compounds (VOCs) and pathogens such as bacteria, microbes, fungi and viruses to increase inside such buildings. This leads to what has been known as sick building syndrome [1]. Not only is the increased level of pollutants and pathogens unhealthy for occupants in such buildings but it also leads to poorer productivity. This has lead to the design of “green” buildings that allow/control interior air turnover rates.

A recent study [2] has shown that both increased levels of carbon dioxide and VOCs leads to measurable cognitive impairment for occupants, recommends increased building ventilation rates and argues that increased heating and cooling costs associated with greater air turnover rates to reduce VOC and carbon dioxide levels is more than offset by increased occupant productivity.

Several recent patents [3-19] address this problem and do so by incorporating a scrubbing unit containing a CO2 sorbent that is operated cyclically (discontinuously), in a sorption mode followed by a desorption mode, where sorbed CO₂ is purged from the scrubbing unit and vented. For example, U.S. Pat. No. 9,328,396 and related patents disclose using gas scrubbing units to remove one or more gases, including CO₂, by adsorption, from the conditioned air stream. However, the scrubbing units are operated discontinuously. A limitation of this approach is that, during purge cycles, CO₂ levels can periodically spike, as illustrated schematically in FIG. 1, which shows how the CO₂ concentration within the working or living spaces of buildings may vary over time.

So, while the average CO₂ concentration can be below a preset safe threshold level, there will be cyclical periods where it may exceed it causing an unhealthy and cognitively impaired environment for the building occupants. It would be a better and healthier approach to provide a more uniform, safe level of CO₂ within the building environment rather than a potentially unhealthy, cyclical and variable level of CO₂.

It would therefore be advantageous to provide more uniform, safe levels of CO₂ within the building environment, rather than a potentially unhealthy cyclical variable level of CO₂ as fostered by existing technology. It would also be advantageous to provide alternative methods for reducing both VOCs and interior carbon dioxide levels that is more energy efficient than increasing ventilation rates.

### SUMMARY

In various embodiments described herein, systems and methods for removing CO₂ and volatile organic compounds (VOCs) from air in a building or other enclosure are disclosed.

In one embodiment, the systems provide a combination of a continuous carbon dioxide scrubber and an air purifier, which in one aspect, is located within a heating, ventilation and cooling (HVAC) system.

The HVAC systems include heating and/or cooling coils, supply air ductwork which provides air to the living and working spaces within a building or other enclosure, and return air ductwork for returning air from the living and working spaces within a building or other enclosure. A carbon dioxide scrubber and air purifier can be located at various positions throughout the HVAC system, and can be used to remove carbon dioxide and VOCs from the air.

In some embodiments, the systems also include particulate removal systems, such as air filters.

Although other air purification systems can be used, in one aspect, the systems comprise one or more cold plasma based ion generator units, which are combined with one or more continuous carbon dioxide removal units. In one aspect of this embodiment, the devices are installed within the recirculating air system of an interior living or working space.

Cold plasma ion generators, such as those made by Top Product Innovations, reduce both VOC levels and pathogen levels within residential and commercial living spaces, and provide an overall feeling of air freshness.

A schematic of a typical HVAC system that embodies the device described herein is shown in FIG. 2. As shown in FIG. 2, a typical HVAC system includes heating and cooling coils (40) supply air ductwork (50) which provides air to the living and working spaces within a building, and return air ductwork (60) for returning air from the living and working spaces within a building. In one embodiment of the device described herein, the HVAC system also includes a carbon dioxide (CO₂) scrubber (10), a filter for removing particulates (20), and an air ionizer (30) which are positioned in-line with each other, before the heating and cooling coils, and in the path of make-up air. The carbon dioxide scrubber is capable of continuously removing carbon dioxide, the air filter can remove particulates, and the air purifier, such as an air ionizer, can remove VOCs, pathogens and other contaminants from the recirculating air. The heating and cooling coils (40) render the air temperate.

The various components can be configured in a different order than illustrated in FIG. 2 if desired. For example, the filter can be placed before the CO₂ scrubber, after the air purifier, after the heating cools, or anywhere along the path of the recirculating air within the ductwork. The CO₂ scrubber can be placed after the filter, after the air ionizer, or after the heating and cooling coils. The air purifier can be placed before the CO₂ scrubber, before the filter, or after the heating and cooling coils.

In one embodiment, the recirculating air of the building interior is blown through a continuously operating CO₂ removal unit based on a falling curtain of droplets of a CO₂ sorbent/dissolving liquid formulation, as shown schematically in FIGS. 3 and 4.

The droplets now containing dissolved CO₂ fall into a collection chamber or vessel, from which they are pumped into a heated pipe or vessel that includes an outlet port which extends outside the building or other enclosure. The CO₂ absorbing liquid formulation is such that, as is typically the case, the CO₂ solubility within it decreases with temperature. Thus, as the liquid is heated, CO₂ is driven out of the heated liquid and vented outside of the building. The CO₂ sorbent/dissolving liquid formulation can then then be cooled and converted back to the falling droplet curtain liquid, whereby CO₂ can be sorbed from the air stream.

Alternatively, or additionally, the liquid containing dissolved/sorbed carbon dioxide can be subjected to a reduced pressure as a means for removing carbon dioxide from it. The unit is operated on a continuous basis at least for so long as the building is occupied or such operation is desired.

The CO₂ sorbent/dissolving liquid comprises any liquid or combination of liquids and dissolvable or dispersed solids or dispersed immiscible liquids in emulsified form with an affinity for carbon dioxide. Examples include, but are not limited to, glycerol, aqueous glycerol, glycerol in combination with amines or polymers comprising amine groups, such as polyDADMAC (polydiallyldimethylammonium chloride), amines, ionic liquids, and combinations thereof.

Preferred CO₂ dissolving liquid formulations have an essentially zero or low vapor pressure under the prevailing conditions.

In another embodiment, a solid CO₂ sorbent, in particulate form, is dispersed and entrapped within a porous felt such as a non-woven fabric. The CO₂ sorbent is preferably bonded to the fabric by some means to avoid loss over time. The fabric is formed into a rotating f as shown schematically in FIG. 5. The circulating air passes through the fabric allowing the absorption of CO₂. The fabric then passes into a heated region whereby CO₂ is desorbed from the fabric and vented outside of the building.

In an embodiment similar to that shown in FIG. 5, containers of a sorbent are present on a conveyor belt, and conveyed between a sorbing position and a desorbing position. In the sorbing position, an air stream with relatively high concentrations of CO₂ is passed through the sorbent containers, providing an air stream with relatively lower concentrations of CO₂. The sorbent is then regenerated by passage through a desorbing position, where one or more of heated air, reduced pressure, or other desorbing conditions is used to remove sorbed CO₂ from the sorbent. By using a plurality of containers, some of which are in the sorbing position while others are in the desorbing position, the process operates in a continuous manner.

In another embodiment, two or more CO₂ removal units are operated in a manner such as that when one or more of them is being used to remove CO₂ from the building air stream at least one other is being regenerated for reuse. Such CO₂ absorbing units can be in fixed positions with means to direct the airflow as required such as with baffles or the CO₂ absorbing units can be moved into the air stream by any appropriate mechanical means, such as valves.

The invention is not limited to the embodiments described above but can be practiced with any means of CO₂ removal that can be operated on a continuous basis.

### BRIEF DESCRIPTION OF THE DRAWINGS

FIG. 1 is a schematic illustration of carbon dioxide levels over time as a prior art discontinuous carbon dioxide removal system is employed.

FIG. 2 is a schematic illustration showing a heating, ventilation, and air conditioning (HVAC) system that includes continuous carbon dioxide removal, and an air ionizer to remover volatile organic compounds.

FIG. 3 is a schematic illustration of a carbon dioxide removal system, wherein a carbon dioxide (CO₂) dissolving liquid is passed through porous baffles, and a stream of air with carbon dioxide in it is passed through the stream. Air with less carbon dioxide is then produced, and the CO₂ dissolving liquid is heated, for example, by heating, to desorb carbon dioxide, at which point the liquid is recirculated.

FIG. 4 is a schematic illustration of a carbon dioxide removal system, where air with relatively high levels of carbon dioxide is passed through a carbon dioxide dissolution section including a carbon dioxide dissolving liquid, to provide air with relatively lower levels of carbon dioxide. The liquid is transferred to holding tanks, and treated, such as by heating, to remove carbon dioxide. The carbon dioxide dissolving liquid is then recycled.

FIG. 5 is a schematic illustration of a carbon dioxide removal system, where air with relatively high levels of carbon dioxide is passed through a substrate which removes carbon dioxide, which substrate circulates, for example, by rotation. The air, after passing through the substrate, has relatively lower levels of carbon dioxide. When the substrate is not in the path of air, it can be treated by heating it with heated air to dissociate carbon dioxide and regenerate the substrate. The resulting heated air, which has relatively higher levels of carbon dioxide due to the carbon dioxide dissociated from the substrate, is then sent to an outside vent.

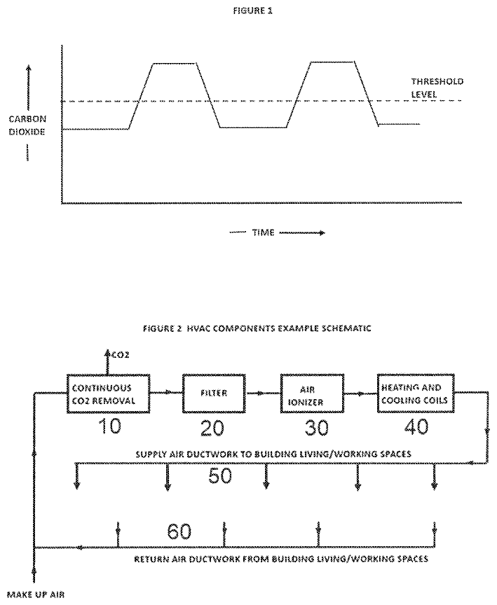

### DETAILED DESCRIPTION

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10,” “from 5 to 10,” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

In the embodiments described herein, devices and methods for their use are provided for purifying air and removing CO₂ and volatile organic compounds (VOCs) and/or pathogens.

The devices include a combination of an air purifier capable of removing volatile organic compounds, and a carbon dioxide scrubber (i.e., a device that reduces carbon dioxide (CO₂) levels). The air purifier and carbon dioxide scrubber are incorporated into an HVAC system, and used to reduce the level of CO₂ and VOCs within a building or other enclosed environment, ideally to levels below those that lead to cognitive impairment of the occupants in the building or other enclosure.

Make up air, typically drawn from the outside environment, can be used for several purposes. In one aspect, it replaces air lost through leakage. In another aspect, if the air within the building is too hot and the outside is cooler, cooler air can be drawn from the outside, thus reducing the cooling load on the air conditioning system. If the air within the building is too cool and the outside is warmer, then warmer air can be drawn from the outside, thus reducing the heating load on the heater.

If the air within the building is unhealthy, containing for example too high a level of carbon dioxide, VOCs and/or pathogens, then outside air can be drawn in to dilute the level of CO₂ and other contaminants to a healthier level. This requires exhausting at least a portion of the contaminated, but thermally conditioned air within the building, and replacing it with outside air that may need to be heated or cooled to maintain the proper temperature within the building. This significantly adds to the cost of operating the HVAC unit(s).

In various embodiments described herein, systems and methods are provided for removing such unhealthy components, without the need to bring in as much outside air, or, in some aspects, any outside air.

In some aspects, the methods involve using a combination of cold plasma ionization unit or units, or absorption units based on, for example, activated carbon, to remove VOCs and pathogens, and carbon dioxide is removed on a continual rather than an intermittent basis.

The embodiments described herein therefore contribute to the health of the building occupants, and reduce the operational cost for the HVAC system for thermally conditioning the air within the building. A further benefit is that it avoids, or at least limits, the potential introduction of pollutants and contaminants from the outside.

### I. Environments to be Treated

Representative enclosed environments include an office building, a commercial building, a bank, a residential building, a house, a school, a factory, a hospital, a store, a mall, an indoor entertainment venue, a storage facility, a laboratory, a vehicle, an aircraft, a ship, a bus, a theatre, a partially and/or fully enclosed arena, an education facility, a library and/or other partially and/or fully enclosed structure and/or facility which can be at times occupied by equipment, materials, live occupants (e.g., humans, animals, synthetic organisms, etc.) and/or any combination thereof.

The enclosed environment can include a plurality of indoor spaces, such as rooms, cubicles, zones in a building, compartments, railroad cars, caravans or trailers, for example. Adjacent to the indoor space can be an air plenum, typically located above the ceiling of the indoor space. Each indoor space can be associated with a separate air plenum, though a common air plenum can be associated with a plurality of indoor spaces. The enclosed environment can optionally include a single indoor space.

The gas within the environment to be treated may include, in addition to carbon dioxide, volatile organic compounds, sulfur oxides, radon, nitrous oxides or carbon monoxide.

Indoor air typically comprises a relatively low concentration of CO₂, ranging from 400-2000 ppm, though this concentration increases in enclosed spaces as occupants respire and fresh air is not circulated within the building. The concentration of CO₂ in outdoor air, external to the enclosed environment, is typically in the range of 300-500 ppm, and is typically lower than within the building. In some embodiments the concentration of CO₂ in outdoor air is lower than in the indoor air by a range of 100-2000 ppm, and in other embodiments, the concentration is lower than in the indoor air by 1200 ppm or less, such as by 800 ppm or less or 400 ppm or less.

As described in more detail below, a sorbent is used to reduce CO₂ levels within the indoor air. Indoor air also includes other gas compounds, in concentrations of around 75-82% or 79-82% nitrogen; and 15-21% or 18-21% oxygen. Water is also present. The amounts vary depending on the humidity level of the indoor air, but levels are typically around 0%-5% water by volume in the indoor air. In some embodiments, it is desirable to use sorbents which selectively capture CO₂, and possibly other substances, but do not sorb water, nitrogen or oxygen.

As used herein, the term “sorption” is intended to encompass both adsorption and absorption.

As used herein, adsorption is the adhesion of atoms, ions or molecules from a gas, liquid or dissolved solid to a surface. This process creates a film of the adsorbate on the surface of the adsorbent. As used herein, absorption is where a fluid (the absorbate) is dissolved by or permeates a liquid or solid (the absorbent), respectively. Adsorption is a surface phenomenon, while absorption involves the whole volume of the material. The term sorption encompasses both processes, while desorption is the reverse of it.

### II. HVAC System

HVAC systems include means for cooling and/or heating air, and for circulating it throughout a given area or building, such as a residential or commercial building or a particular environment within the same.

In one embodiment, the system includes a heater, wherein the received outdoor air is heated by the heater. Examples of suitable heaters include heat pumps, electric heating coils, coils or radiators with heated fluid supplied from a central heating system, solar heaters, and furnaces. The heat pump may remove heat from the indoor air.

The heated outdoor air can be heated before or after being supplied to the scrubber.

Typically, conditioned air travels to the sections or rooms within the building by a series of ducts from which it is vented into the required rooms or areas through registers. Return registers funnel the air back through another network of ducts to the heating or cooling unit or units.

A distributed air circulation system conveys chilled or heated fluid to local air circulation units. Typically, nearly each indoor space is associated with a local air circulation unit, which circulates and cools or heats the indoor air of the indoor space. Each indoor space can be associated with an air circulation unit. As discussed in more detail below, a CO₂ scrubber and air purifier are provided as part of the HVAC system, and can be arranged adjacent to or within the indoor space.

The air circulation unit can include a fan-coil unit, or any other suitable device for circulating and cooling or heating air in indoor spaces, such as a blower-coil unit, for example. In some embodiments, the air circulation unit can be a component in a split unit system.

In some embodiments, the HVAC system includes a fan-coil unit arranged adjacent to or within an indoor space within the building and additionally configured to heat and/or cool the air of the indoor space. In some aspects of these embodiments, the fan-coil unit may include housing including a fan and coils, and wherein at least a portion of the scrubber can be housed within the housing. A fan of the fan-coil unit can be configured to direct indoor airflow into the scrubber. The coils are typically cooled or heated by a fluid. The coils can include a cooling coil and/or a heating coil and/or any other suitable cooling or heating means, such as radiators, electrical heaters, chillers, heat exchangers, nozzles, jets, and the like.

The chilled or heated fluid may originate from a centralized chilling or heating system shared by a plurality of air circulation units, or from a single dedicated heat pump or boiler.

In some embodiments, the fluid can be supplied by a Variable Refrigerant Flow (VRF) system. In other embodiments, the fluid is supplied by a Fixed Refrigerant Flow system or by a direct expansion (DX) system. In some distributed air circulation systems, the fluid is water.

At least a portion of the indoor air may exit the indoor space as return air. In accordance with some embodiments, the return air may enter the air plenum. Typically, the return air enters the air plenum without flowing through a duct, though in some embodiments a duct is provided.

In other embodiments, the indoor space can be associated with an adjacent area above its ceiling instead of the air plenum. The return air may flow within a duct located in the area above the ceiling to the fan-coil unit.

The fan draws the return air to enter fan-coil unit, via an entry port, and flow in the vicinity of coils for heating or cooling thereof. The coils can be placed downstream the fan. Alternatively, the coils can be placed intermediate the fan and the entry port or at any other suitable location. Return air may flow through a particle filter for removing dust and airborne particles therefrom.

Conditioned air, i.e. return air cooled or heated by the coils, exits via an exit port. The conditioned air enters the indoor space for circulation thereof. The conditioned air may flow from the fan-coil unit into the indoor space via a duct or may ductlessly flow into the indoor space.

A portion of the indoor air can be exhausted from the enclosed environment as exhaust air into the ambient or any location outside the enclosed environment. Any suitable means, such as a blower or a fan can be used to exhaust the exhaust air. The exhaust air may exit the indoor space, via an exhaust port, and/or may exit the air plenum, via an exhaust port or via an exhaust port of the fan-coil unit.

In standard distributed air circulation systems fresh, outdoor air or namely “makeup air” can be introduced into the enclosed environment for supplying nominally fresh, good quality air combining with the return air. The outdoor air can be introduced into the enclosed environment in any suitable manner, such as by a network of ducts. Outdoor air can be introduced directly into each of the indoor spaces, via an entry port, or outdoor air can be introduced into the air plenum, via an entry port. In another embodiment, the outdoor air can be introduced directly into each fan-coil unit. The duct is directed to introduce the outdoor air into the fan-coil unit prior to the particle filter, though the outdoor air can be introduced into the fan-coil unit at any suitable location therein.

Details of HVAC systems for residential and commercial use are freely available. One representative example of an overview of HVAC systems can be found at: https://www.energycodes.gov/sites/default/files/becu/HVAC_Systems_Presentation_Slides.pdf.

### III. Air Purifiers

The systems described herein include at least one additional air treatment component (air purifier) suitable for removing volatile organic compounds and/or pathogens. Representative air purifiers include air ionizers, an ozone source, a source of radiation, a membrane, foam, paper, fiberglass, a particle filter, an ultraviolet anti-microbial device, an ion or plasma generator, such as a cold plasma-based ion generator unit, and a catalyst, such as an oxide catalyst and/or or a chemical catalyst.

The air purifier component can be placed within the HVAC system, for example, adjacent to a CO₂ scrubber, in line with the scrubber, or elsewhere in the path of air to be purified. The additional air treatment component can be placed within the indoor space. For example, the indoor air can flow out of the fan-coil unit via a duct, and an additional air treatment component can be placed within the duct.

Representative air purifiers include, but are not limited to, air ionization units, activated carbon adsorption units, and combinations thereof. Suitable air ionization units which can be used in the systems described herein include, but are not limited to, air ionization units manufactured by Top Product Innovations of NC.

The general concept of using bipolar ionization to generate positive and negative ions or free radicals that find application in air purification systems goes back to the 1960s. Since that time, there have been numerous DC, AC, and pulsed waveform bipolar generators, any of which can be used in the systems described herein. Any cold plasma ionization unit that can be used to remove volatile organic compounds from air can be used in the systems described herein.

In operation, very typically, oxygen and other components of air, such as moisture vapor, are ionized to produce transient negative, positive ions, radicals or energetic species that can interact with and destroy volatile organic compounds (VOCs), odor molecules and pathogens of all types present in the air or present on surfaces.

In a preferred embodiment, the ionization energy used in the cold plasma ionization unit or units is controlled to be no more than 12.6 electron volts (eV) to minimize or eliminate ozone production.

While not wishing to be bound to a particular theory, it is believed that the ions produced by ionization break down volatile organic compounds in the air to harmless compounds prevalent in the atmosphere, such as oxygen, nitrogen, water vapor and carbon dioxide.

These compounds are formed after volatile organic compounds enter into the plasma field. By way of example, formaldehyde is frequently emitted by building materials, for example, orient strand board (OSB) held together with urea-formaldehyde resin. When formaldehyde is exposed to a plasma field, it breaks down to carbon dioxide and water vapor, thus eliminating health hazards associated with formaldehyde exposure. Another example is ammonia and other amines, which can be produced by building occupants, and these amines typically break down to oxygen, nitrogen and water vapor upon exposure to a plasma field. As you can see, what chemical you start with determines how it reacts with the ionization field and how it breaks down.

In addition to the plasma reacting with volatile organic compounds, the positive and negative ions are also drawn to airborne particles by virtue of their electrical charge. Once the ions attach to the particle, the particle grows larger by attracting nearby particles of the opposite polarity, which makes it easier to remove particulates from the air.

Similarly, positive and negative ions also attracted to pathogens, such as mold, and other microbes. When the ions combine on the surface of a pathogen, they inhibit the ability of the pathogen to thrive, making it possible to lower the amount of airborne viruses, bacteria and/or mold spores in the air.

Alternatively, or additionally, VOC absorption units based on activated carbon or other absorbents, or mixtures of the same, and other filtration devices to remove particulates from the air, can be used, alone, or in addition to, cold plasma ionization units.

### IV. Carbon Dioxide Scrubbers

The air treatment systems described herein include one or more carbon dioxide (CO₂) scrubbers configured to scrub air from one or more indoor spaces within the building, and can be positioned within or adjacent the one or more indoor spaces.

In one embodiment, a plurality of indoor spaces is provided with a plurality of fan-coil units. The plurality of fan-coil units are arranged adjacent to or within the plurality of indoor spaces, and one or more scrubbers can be configured to scrub indoor air from the plurality of indoor spaces.

Each CO₂ scrubber is configured to scrub indoor air from the indoor space. The scrubber includes one or more sorbent materials, which can be adsorbent materials, absorbent materials, or combinations thereof. The sorbent materials are arranged therein to sorb CO₂, and possibly other contaminant gases, from one or more of the indoor air, a source of outdoor air, and/or an exhaust.

Suitable CO₂ sorbents include any liquid, combination of liquids, dissolvable or dispersed solids, or dispersed immiscible liquids in emulsified form, that have an affinity for carbon dioxide. Examples include, but are not limited to, glycerol, aqueous glycerol, and glycerol in combination with amines or polymers comprising amine groups, such as polyDADMAC (polydiallyldimethylammonium chloride).

In the embodiments described herein, the scrubber is intended to be used continuously, and not cycled. In some aspects, the sorbent travels along a path, where it contacts an airflow at one position on the path (a scrubbing position), and sorbed carbon dioxide is desorbed at another position (a purging position) on the path, after which the sorbent is recycled through the path.

The airflow to be scrubbed is typically indoor air, and once the sorbed carbon dioxide is purged from the sorbent, it is typically directed outside of the building, though can optionally be stored for later use or sale.

Outdoor air can be passed over and/or through the sorbent materials to purge, or desorb, at least a portion of the CO₂ sorbed by the sorbent materials, Desorbed CO₂ can be removed from the building, for example, via the exhaust.

Following the capture of the substances, the sorbent construct is regenerated by urging the release of at least a portion of the sorbed CO₂. Regeneration is a very important aspect of sorbent performance and often the step where the most energy is required.

Sorbents can be regenerated by one or more of heating, purging, pressure change, electrical energy, and combinations thereof. Additionally, carbon dioxide can be desorbed using a combination of heating and purging with air or another purge gas. The released substances can be expunged into the atmosphere or otherwise collected, disposed of, sequestered, and/or any combination thereof.

In some embodiments, the scrubber supplies scrubbed air exiting the scrubber to the indoor space, for example, by supplying scrubbed air to a fan-coil unit arranged adjacent to or within the indoor space. In some embodiments, the flow of indoor air from the indoor space to the scrubber or from the scrubber to the indoor space is ductless.

Sorbents and Sorbent Constructs

A sorbent, by itself or present in a “sorbent construct,” can reduce the concentration of carbon dioxide, and, optionally, other contaminants present in the indoor air. A sorbent construct includes a sorbent that is composed of at least two functional groups of materials: a passive support and an active compound. The support materials generally provide the mechanical and physical structure of the sorbent and the active compound attracts and captures CO₂.

Carbon dioxide sorbents include sorbents in liquid or solid form. Suitable sorbent materials include granular adsorbent particles, solid supported amines, activated carbon, clay, carbon fibers, carbon cloth, silica, alumina, zeolite, synthetic zeolite, hydrophobic zeolite, natural zeolite, molecular sieves, titanium oxide, polymers, porous polymers, polymer fibers or metal organic frameworks. At least one of the sorbent materials can be contained in one or more removable cartridges. Certain of these sorbents also remove volatile organic compounds, in addition to carbon dioxide.

Supports

The support component of the sorbent can be formed of any suitable material. In a non-limiting example the support can be formed of generally chemically inert materials, and is preferably a porous support, as relatively porous supports tend to have relatively high surface areas.

Examples of chemically inert supports with a large total surface include clay, silica, metal oxides like alumina, and combinations thereof. Examples of these materials include zeolites, porous alumina, porous minerals, silica, porous silica, silica nanoparticles, fumed silica, activated charcoal and metal organic frameworks, for example. Representative clays include aluminum phyllosilicates such as bentonite, montmorillonite, ball clay, fuller's earth, kaolinite, attapulgite, hectorite, palygorskite, saponite, and sepiolite.

Additionally, the support can be formed of sorbent materials, such as gels, molecular sieves, nanotube-containing materials, porous materials, fiber based materials, sponge-like materials, electrically and/or electro-magnetically charged liners or objects, porous organic polymers, and combinations thereof.

Clays and other supports can bind with amines to form a sorbent support, as disclosed, for example, in U.S. Pat. No. 6,908,497 to Siriwardane, where bentonite clay is impregnated with an amine, resulting in amine impregnated clay that acts as a selective adsorbent of CO₂.

Acid-treated bentonite clay chemically binds well with the hydroxyl group of diethanolamine (DEA) to form a stable sorbent, and polyethylenimines (PEI) attach to silica surfaces. These combinations of supports and amines can be used as sorbents for selective and efficient CO₂ removal from indoor air.

In some embodiments, the support may comprise a combination of several different sorbent materials. The support can be formed in any suitable configuration, such as a solid supporting substrate or solid support formed with a relatively large total surface area. The solid support may comprise any suitable material that is not a liquid. For example, the solid support can be formed of a plurality of elements such as solid particles 130 or sheets, for example. The total surface area can be generally defined as the sum of the surface areas of each element forming the solid support.

The particles can be configured in any suitable shape or method such as powders, fibers, granules, beads, pellets, extrudates or a combination thereof. The fibers can be any suitable fiber such as carbon fiber, silica fibers or polymer fibers, for example. The fibers can be weaved or intertwined to form a fabric or a paper-like material.

The fibers, granules, beads, pellets and extrudates can be formed of any suitable material as described hereinabove. The support can be formed of a plurality of thin sheets. The sheets may comprise natural or synthetic fiber based materials, paper, natural fabrics, or synthetic fabrics. The sheets can be formed in any suitable size, such as with a thickness in the range of approximately one micron to two centimeters. Additionally, the range can be approximately 2-80 millimeters, for example. In some embodiments the sorbent may comprise a large surface area (such as the total surface area of the plurality of particles), for example in the range of 10-1000 square meters per gram.

The particles can be formed with dimensions ensuring that the particles are not too fine thereby forming an overly dense layer, which may prevent the flow of the indoor air through the sorbent construct. Additionally, the particles can be formed with dimensions ensuring that a total surface area of the plurality of particles is sufficiently large for allowing the indoor air to have maximal contact with the particles for maximal adsorption of the substance, such as the CO₂.

In a non-limiting example, the average diameter of the particles can be in the range of approximately 0.1-10 millimeters. Not all particles are likely to be identical in shape and size, therefore the typical or average particle comprises an average of an aggregate of such particles. In another non-limiting example, the average diameter of the particles can be in the range of approximately 0.2-3 millimeters. In yet another non-limiting example, the diameter of a particle can be in the range of approximately 0.3-1 millimeters. The diameter of the particle can be measured as the approximate diameter, wherein the particle is a granule or bead, or can be measured as a cross section diameter, wherein the particle is a fiber, an extrudate or a pellet.

In a non-limiting example, a solid support can be impregnated by the amine-based compound in any suitable manner, such as by spraying, dripping or immersion within a solution of the amine-based compound, for example. The impregnation can additionally, or alternatively, be mechanically stimulated, with catalysts, or with external energy sources, such as heat.

In accordance with some embodiments, the solid support can initially comprise particles, thin sheets or any configuration which provides a relatively large total surface area for contact with substances, which are thereafter impregnated by an amine-based compound and/or an ionic liquid.

This amine- or ionic liquid-containing solid sorbent allows the indoor air to have maximal contact with the sorbent for maximal CO₂ removal from the indoor air. This is advantageous for indoor air which typically has a low concentration of CO₂. Additionally, use of sorbent-containing supports with a relatively large total surface area allows one to dispose the sorbent construct within a relatively compact, small sized enclosure.

In accordance with other embodiments, the solid support may initially comprise fine particles, such as a powder. The fine particles can be mixed with the amine-based compound, such as by immersion of the fine particles in an amine-containing liquid or solution to form an amine-containing powder.

In accordance with other embodiments the amine-containing powder is agglomerated to form the support by standard procedures, such as granulation for forming granules or beads and pelletization or extrusion for forming pellets or extrudates.

Amine-based sorbents can be a liquid, a solid or can be initially a solid dissolved in a solvent. For example, an amine-based compound such as diethanolamine (DEA) or polyethyleneimine (PEI) can be dissolved in any suitable solvent, such as dichloromethane (DCM), water, ethanol, methanol, ethylene glycol (EGW) or propylene glycol (PGW), for example. Supports can then be contacted with solutions of the sorbents, and the solvents removed, to provide sorbent constructs.

The conditions and parameters for forming the amine-containing support may vary according to the properties of the selected support and the selected amine-based compound.

The sorbent constructs can be placed within the enclosed environment. Alternatively, the sorbent constructs can be placed out of the enclosed environment and the indoor air and/or the outside air can be introduced therein in any suitable manner.

As described hereinabove, the sorbent can be formed of porous materials thereby allowing the indoor air to also flow through the particles or sheets and thus enhancing the gas permeability of the sorbent.

Moreover, the sorbent structured with a relatively large total surface area and with relatively high fluid permeability provides for relatively rapid reaction kinetics and thus a relatively large quantity of CO₂ is captured quickly by the amine-containing support. Reaction kinetics or chemical kinetics can be defined as the rate of a chemical process, such as the rate the CO₂ is captured by the sorbent.

The sorbent/sorbent construct can be arranged in any suitable manner. For example, the sorbent can be placed within an enclosure formed in any suitable configuration. The particles or thin sheets or any other sorbent construct can be relativity densely packed within the enclosure at a density allowing the indoor air to have maximal contact with the particles for maximal interaction therefrom yet not overly dense, which may prevent the flow of the indoor air through the sorbent construct.

Low flow resistance and rapid reaction kinetics is advantageous for capturing CO₂ from indoor air with a relatively low CO₂ concentration.

Low flow resistance and a relatively large total surface area of the sorbent also allows the purge air to readily release sorbed CO₂ from the sorbent, as explained hereinabove. Consequently, the purge air may regenerate the sorbent with minimal added heat in a relatively short time, and, additionally the purge air may comprise outdoor air.

Sorbents

Representative carbon dioxide sorbents include glycerol, amines, ionic liquids, and combinations thereof. Amines can be liquid or solid, ionic liquids are in the liquid phase, and glycerol is a liquid.

Mixtures of different dimethyl ethers and polyethylene glycol, represented by the formulae (CH₃O(C₂H₄O)ₙCH₃), with n ranging from 3 to 9 or thereabouts, including those under the tradename Selexol™ can also be used, as can propylene carbonate, alone or in admixture with other sorbents.

In one embodiment, the sorbent is an amine-based or amine-like compound. The amine compound is suitable for adsorbing CO₂ present in the indoor air. Amines can selectively capture a relatively large amount of CO₂. In accordance with some embodiments the amine-based compound may comprise a relatively large fraction of secondary amines. In some embodiments like diethanolamine, the amines are 100% secondary amines. In other embodiments, like certain polyamines, between 25%-75% of amines are secondary amines. Additionally, the amine-based compound may comprise at least 50% secondary amines. Moreover, the amine-based compound comprises at least 25% secondary amines.

The amine-based compound can be, or comprise, any suitable amine, such as a primary or secondary amine, or a combination thereof. Additionally, the amine-based compound may range from relatively simple single molecules, such as ethanolamine, to large molecule amino polymers such as polyethylenimine. The amine-based compound may comprise monoethanolamine (MEA), ethanolamine, methylamine, branched polyethyleneimine (PEI), linear polyethyleneimine (PEI), diethanolamine (DEA), dimethylamine, diethylamine, diisopropanolamine (DIPA) tetraethylenepentamine (TEPA), methyldiethanolamine (MDEA), methylethanolamine, and any of a number of polyamines such as polyethylenimine, or a combination thereof, for example.

Primary amines, which comprise NH2 elements, create strong chemical reaction with CO₂. Secondary amines, which comprise a single hydrogen, i.e. NH, create weaker chemical reaction with CO₂ yet still efficiently and selectively capture the CO₂. Accordingly, secondary amines require less energy for releasing the captured CO₂ therefrom than the energy (such as thermal energy) required for releasing CO₂ from primary amines. The more-readily releasing secondary amines allow satisfactory capturing of the CO₂ from indoor air while also allowing relatively rapid and low energy regeneration of the sorbent. Accordingly, sorbent regeneration can be performed at relatively low temperatures using outdoor air. The ability to regenerate the sorbent with minimal added heat is highly advantageous for treating indoor air.

Examples include but are not limited to glycerol, aqueous glycerol, or aqueous glycerol, glycerol combinations with amines, such as primary amines such as monoethanolamine (MEA) and diglycolamine (DGA), or secondary amines such as diethanolamine (DEA) and diisopropanolamine (DIPA) or tertiary amines such as triethanolamine (TEA) or methyldiethanolamine (MDEA) or amino group containing polymers such as polyDADMAC admixed therein. Preferred CO2 dissolving liquid formulations have an essentially zero or low vapor pressure under the prevailing conditions.

While amines, such as monoethanolamine and other amines discussed above, are commonly used in carbon capture applications, amines tend to be somewhat corrosive, degrade over time, and have volatility. Ionic liquids on the other hand, have low vapor pressures, and their vapor pressure remains low through their thermal decomposition point (typically >300 ° C.). This low vapor pressure simplifies their use, and makes them more “green” alternatives to amines. Additionally, the use of ionic liquids can help reduce the risk of contamination of the CO₂ gas stream and of leakage into the environment.

Ionic liquids have also been developed to sorb carbon dioxide. Amino acid ionic liquids, such as those based on lysine, are capable of sorbing carbon dioxide (see, for example, Firaha and Kirchner, “Tuning the Carbon Dioxide Absorption in Amino Acid Ionic Liquids,” Chemistry and Sustainability, Volume 9, Issue 13, Pages 1591-1599 (2016)).

Ionic liquids are essentially zero vapor pressure CO₂ dissolving liquids, and have recently been reviewed by Zeng [20] and Luo [21]. Further details of CO₂ dissolving liquids operating by either chemical or physical absorption processes are reviewed in detail by Vega et al [22]. These references, as with all other references discussed herein, are incorporated by reference in their entirety for all purposes.

The solubility of CO₂ in ionic liquids is governed primarily by the anion, and less so by the cation, with the hexafluorophosphate (PF6-) and tetrafluoroborate (BF4-) anions being especially amenable to CO₂ capture (see, for example, Ramdin, et al., “State-of-the-Art of CO₂ Capture with Ionic Liquids,” Industrial & Engineering Chemistry Research. 51 (24): 8149-8177 (2012)). Representative ionic liquids include 1-butyl-3-propylamineimidazolium tetrafluoroborate, 1-ethyl-3-methylimidazolium (EMIM), and ionic liquids with cations like trihexyl(tetradecyl) phosphonium.

Ionic liquids for carbon capture tend to have relatively higher viscosity than amines. Ionic liquids that use chemisorption depend on a chemical reaction between solute and solvent for CO₂ separation. The rate of this reaction is dependent on the diffusivity of CO₂ in the solvent and is thus inversely proportional to viscosity. The viscosity of an ionic liquid can vary significantly according to the type of anion and cation, the alkyl chain length, and the amount of water or other impurities in the solvent. These solvents can be “designed” and these properties chosen, so those of skill in the art can develop suitable ionic liquids with lowered viscosities, and/or support the ionic liquids as “supported ionic liquid phases,” or (SILPs).

Scrubbers Including Sorbents and/or Sorbent Constructs

The scrubbers comprise one or more sorbents and/or sorbent constructs. In some embodiments, the scrubbers further comprise at least one of a damper and a fan.

Whereas scrubbers used in prior art systems have discontinuous scrub cycles and purge cycles, the scrubbers described herein are designed to be run continuously. Air with relatively higher amounts of carbon dioxide is scrubbed by passing it through a sorbent, optionally present within a sorbent construct, which in some embodiments is liquid, and in other embodiments is solid, to produce an air stream with relatively lower amounts of carbon dioxide.

Different scrubbers are used depending on whether the CO₂ sorbents are solid or liquid. For example, scrubbers including liquid CO₂ sorbents can provide a curtain of a CO₂ sorbent, through which air to be treated is passed, or air can be bubbled through the liquid sorbent. Scrubbers including solid CO₂ sorbents can include sorbents in a woven/non-woven material, such as a web or conveyor belt, or a container, such as a porous container, which includes the sorbents. Air to be treated can be flowed through the web, conveyor belt, and/or porous container.

As discussed in more detail below, In various embodiments, the CO₂ sorbent continuously travels along a path, where it picks up/sorbs/dissolves carbon dioxide from the air passed through it, then moves to a desorption zone where it is treated, such as by heating, to desorb the carbon dioxide, and is then recycled through the path. In this manner, the carbon dioxide removal is continuous.

Sorption/Desorption Processes

A typical CO₂ sorption process, regardless of which sorbent is used, typically involves passing a feed gas through and a sorbent, whether through a column, a curtain of droplets of the sorbent, when the sorbent is a liquid, or through a sorbent construct. The CO₂/sorbent is then subjected to a desorption process, where output streams of CO₂-rich gas are sent outside the building, and air relatively lower in CO₂ concentration is released into the building.

Desorption of both amines and ionic liquids can be performed at elevated temperatures, for example, in a stripper column, where carbon dioxide is desorbed. However, ionic liquids can also be stripped using pressure swings or inert gases, reducing the process energy requirement (Zhang et al., “Carbon capture with ionic liquids: overview and progress”. Energy & Environmental Science. 5 (5): 6668 (2012)).

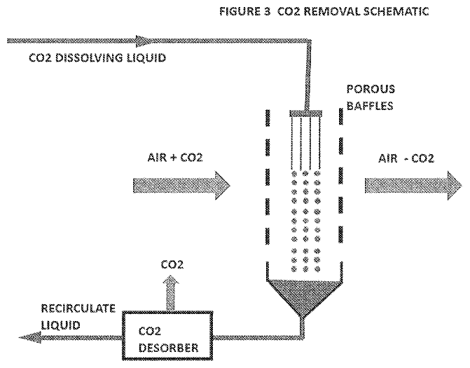

Non-limiting examples of continuous carbon dioxide scrub cycles are shown in FIGS. 3-5.

In one embodiment, the recirculating air of the building interior is blown through a continuously operating CO₂ removal unit based on a falling curtain of droplets of a CO₂ sorbent. The droplets now containing sorbed/dissolved CO₂ fall into a collection chamber or vessel from which they are pumped into a heated pipe or vessel. The CO₂ sorbent is selected such that, as is typically the case, the CO₂ solubility within it decreases with increasing temperature thus CO₂ is driven out of the heated liquid, where it can be vented outside of the building or stored for later use or sale. Alternatively, or additionally, the liquid containing sorbed/dissolved CO₂ can be subjected to a reduced pressure to further facilitate the removal of CO₂ from it. The CO₂ sorbent is then cooled and converted back to the falling droplet curtain liquid whereby CO₂ is once more sorbed from the air stream. This is illustrated schematically in FIG. 3.

To prevent the droplets from being carried away in the air stream, a porous baffle can be installed to collect them and funnel them back to droplet collection point. The porous baffle can, for example be a supported sheet of porous fabric, woven or nonwoven, or wire mesh or multilayer combinations of the same or any other suitable air porous structure. Preferably, the porous baffles comprise outside surfaces that have a sufficiently high surface free energy such that they are fully wetted by the CO₂ sorbent, which minimally impedes the flow of air through them. In the event the CO₂ sorbent dissolves more CO₂ at ambient temperature than a lower temperature, the liquid can be cooled, rather than heated, to desorb the CO₂.

In an alternative configuration, the CO₂ sorbent is not converted into droplets, but rather is metered onto a porous baffle, which it coats as a thin film. Any means of providing a high surface area of CO₂ sorbent, which also resists said liquid film being blown from it and is porous to the airflow required for circulation within the building, is suitable for use in the systems described herein. The relatively high surface area helps to maximize the rate of CO₂ sorption from the impinging air stream within the liquid film. The thinner the liquid film, the more tenaciously it will be held onto the surfaces of the porous baffle. Likewise, relatively more viscous liquids will be less likely to suffer cohesive failure than relatively less viscous liquids, such that droplets are sheared off the liquid film into the air stream. It can also be preferred to use CO₂ sorbents with low or essentially zero vapor pressure under the ambient air conditions to which they are exposed. For this reason, viscous ionic liquids, which have relatively high viscosity and relatively low vapor pressure, can be preferred in this embodiment.

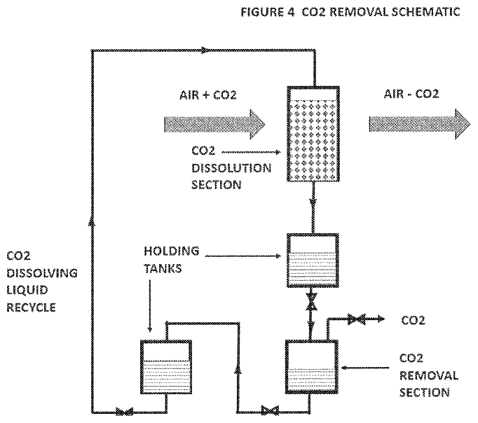

In one embodiment, the CO₂ dissolving liquid is pumped continuously from a main holding tank where in droplet or curtain form it is exposed to the circulating air stream as previously described. Now containing sorbed/dissolved CO₂, the liquid flows into a separate collection vessel. From there, a portion of the CO₂ containing liquid flows or is pumped, for example, via a one way valve, into a second vessel equipped with means to heat the contained liquid and optionally subject it to reduced pressure. This second vessel can be equipped with two further outlets. One is a vent to the outside and the other that leads to the main liquid holding tank. With all valves closed save that to the outside vent, the CO₂ containing liquid in said second vessel can be heated and optionally subjected to reduced pressure, discharging the liberated CO₂ via the vent to the outside. Subsequently the valve to the holding tank can be opened, and the regenerated CO₂ sorbent in the second vessel pumped into the main holding tank. In this manner, the CO₂ is continuously removed from the circulating air in the building and vented to the outside. This process is illustrated schematically in FIG. 4.

In one aspect of this embodiment, the system also includes an automatic system of controls for operating the sorption/desorption process.

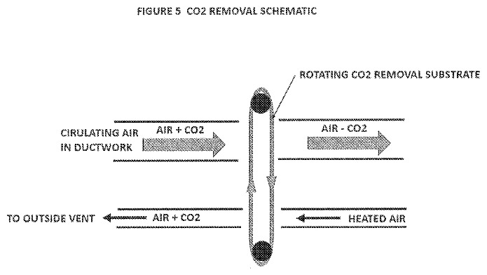

In another embodiment, a solid CO₂ sorbent, in particulate form, is dispersed and entrapped within a porous substrate, such as a non-woven fabric. The CO₂ sorbent is preferably bonded to the fabric by some means, such as through ionic or covalent bonds, adhesion, or mechanical entrapment, to avoid loss over time. The fabric can be formed into a rotating loop or conveyor. The circulating air passes through the fabric, allowing for sorption of CO₂ to take place. The fabric then passes into a heated region whereby CO₂ is desorbed from the fabric, and vented outside of the building. This process is illustrated schematically in FIG. 5.

In another embodiment, two or more CO₂ removal units are operated in a manner such as that when one or more of them is being used to remove CO₂ from the building air stream, at least one other is being regenerated for reuse. Such CO₂ absorbing units can be in fixed positions with means to direct the airflow as required such as with baffles or the CO₂ absorbing units can be moved into the air stream by any appropriate mechanical means.

The CO₂ removal unit is operated on a continuous basis at least for so long as the building is occupied or such operation is desired.

The invention is not limited to the embodiments described above but can be practiced with any means of CO₂ removal that can be operated on a continuous basis rather than an intermittent basis.

In the sorbent station, indoor air with relatively high carbon dioxide concentrations is passed, optionally through ductwork, through the sorbent, where carbon dioxide is sorbed. The resulting air stream includes relatively lower carbon dioxide levels. The sorbent is then passed through a stream of heated air, which desorbs carbon dioxide and regenerates the sorbent. The resulting air, which has a relatively higher carbon dioxide concentration, can then be vented outside the building or other enclosure.

System Controls

While the system is designed to run continuously, it can be shut off, for example, during non-peak hours, weekends, holidays, and the like. The system may further include a controller to switch the system on and off, according to a preset schedule.

The system can also include a monitor or sensor, which can be automatic, or monitored by users, to measure carbon dioxide levels, and turn the system on when a predetermined threshold level of carbon dioxide is measured.

Other data points can be used to signal the system turning on and off, though while in operation, functioning in a continuous manner. Representative data points include, for example, the indoor space occupancy level. The system can optionally include a manual or automatic control for turning the desorption system on or off.

Configuration of an HVAC System Comprising the CO2 Scrubber and Air Purifier

As discussed above, an HVAC system typically includes heating and/or cooling coils, a supply air duct to send air to the building's living and/or working spaces, and return air ductwork from the living and/or working spaces.

As shown in FIG. 2, one representative way to include the CO₂ scrubber and air purifier is to pass return air through a continuous CO₂ scrubber, then optionally through a filter to remove particulates, then through an air ionizer or other air purifier to remove VOCs, before being passed through the heating and/or cooling coils. However, as will be readily apparent, the various components can be configured in a different order than illustrated in FIG. 2 if desired.

The HVAC systems can include any of the embodiments of CO₂ scrubbers described herein, as well as any of the embodiments of air purifiers described herein, as well as optionally further including air filters to remove particulates.

It is to be understood that all the references cited in this disclosure and its attachments are to be incorporated herein in their entirety.

Groupings of alternative elements or embodiments disclosed herein are not to be construed as limitations. Each group member can be referred to and claimed individually or in any combination with other members of the group.

Certain embodiments are described herein. Of course, variations on these described embodiments will become apparent to those of ordinary skill in the art upon reading the foregoing description. The inventors expects skilled artisans to employ such variations as appropriate, and the inventor intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

While the invention has been described with reference to the various preferred embodiments listed herein, it will be understood by those skilled in the art that various changes can be made, and equivalents can be substituted for elements thereof without departing from the scope of the invention. In addition, many modifications can be made to adapt a particular situation or material to the teachings of the invention without departing from the essential scope thereof.

Therefore, it is intended that the invention not be limited to the particular embodiments disclosed as the best mode contemplated for carrying out this invention, but that the invention will include all embodiments falling within the scope of the appended claims.

### REFERENCES

[1] Indian J. Occup. Environ. Med. 2008 August; 12(2): 61-64.
[2] https://www.hsph.harvard.edu/joseph-allen/epa-knowledge-in-the-air/
[3] U.S. Pat. No. 10,281,168
[4] U.S. Pat. No. 10,143,959
[5] U.S. Pat. No. 10,086,324
[6] U.S. Pat. No. 10,046,266
[7] U.S. Pat. No. 9,987,584
[8] U.S. Pat. No. 9,976,760
[9] U.S. Pat. No. 9,950,290
[10] U.S. Pat. No. 9,939,163
[11] U.S. Pat. No. 9,933,320
[12] U.S. Pat. No. 9,919,257
[13] U.S. Pat. No. 9,789,436
[14] U.S. Pat. No. 9,566,545
[15] U.S. Pat. No. 9,533,250
[16] U.S. Pat. No. 9,399,187
[17] U.S. Pat. No. 9,375,672
[18] U.S. Pat. No. 9,328,936
[19] U.S. Pat. No. 9,316,410
[20] Zeng S et al Ionic-Liquid-based CO₂ capture systems. Structure, interaction and process. Chemical Reviews 2017; 117: 9625-9673
[21] Luo X, Wang C. The development of carbon capture by functionalized ionic liquids. Current Opinion in Green and Sustainable Chemistry. 2017; 3:33-38
[22] “Solvents for Carbon Dioxide Capture” by Fernando Vega, Mercedes Cano, Sara Camino,
Luz M. Gallego Fernandez, Esmeralda Portillo and Benito Navarrete
Submitted: Apr. 24, 2017; Reviewed: Oct. 4, 2017; Published: Aug. 16, 2018; DOI: 10.5772/intechopen. 71443, https://www.intechopen.com/books/carbon-dioxide-chemistry-capture-and-oil-recovery/solvents-for-carbon-dioxide-capture

1. A method of operating a heating, ventilation, and air-conditioning (HVAC) system for an enclosed environment, the HVAC system configured to heat and/or cool air, comprising:
circulating, via an air circulation system, air within the enclosed environment comprising indoor air;
introducing, via an outdoor air inlet, outdoor air originating from outside the enclosed environment into the enclosed environment;
continuously removing, via a carbon dioxide scrubber including a sorbent, carbon dioxide from the circulated air;
desorbing, the carbon dioxide from sorbent; and
venting the desorbed carbon dioxide outside of the enclosed environment.

2. The method of claim 1, further comprising:
passing the circulated air through one or more air purification units.

3. The method of claim 2, wherein at least one of the air purification units is an ionization unit.

4. The method of claim 1, further comprising:
passing the circulated air through one or more filtration units to remove particulates.

5. The method of claim 1, further comprising:
passing the circulated air through one or more air ionization units and through one or more filtration units.

6. The method of claim 1, wherein the carbon dioxide scrubber comprises a liquid carbon dioxide sorbent.

7. The method of claim 6, wherein at least some of the carbon dioxide present in the circulated air is sorbed in the sorbent.

8. (canceled)

9. The method of claim 1, wherein the desorbing step is performed by reducing pressure, elevating temperature or bubbling air into the sorbent.

10. The method of claim 1, wherein the sorbent is recirculated.

11. The method of claim 6, wherein the liquid carbon dioxide sorbent is a single liquid, a mixture of liquids, one or more liquids with dissolved solids, one or more liquids with dispersed solids, or dispersed immiscible liquids.

12. The method of claim 6, wherein the liquid carbon dioxide sorbent has a zero or close to zero vapor pressure at an ambient temperature of the building.

13. The method of claim 6, where the liquid carbon dioxide sorbent comprises one or more of glycerol, water, an amine, an amine-containing polymer, and an ionic liquid.

14. The method of claim 1, wherein the carbon dioxide scrubber comprises a carbon dioxide sorbent adhered, attached or entrapped within a porous substrate.

15. The method of claim 14, wherein the porous substrate is configured to move, and wherein a portion of the porous substrate moves to a position adjacent a duct and is exposed to an airstream passing therethrough from which carbon dioxide is sorbed into the porous substrate.

16. The method of claim 15, further comprising:
moving the portion of the porous substrate to another position adjacent another duct where the sorbed carbon dioxide is desorbed from the porous substrate and vented via the other duct outside the enclosed environment.

17. The method of claim 1, wherein the carbon dioxide sorbent is adhered, attached or entrapped within a porous substrate, and wherein the HVAC system includes plural carbon dioxide scrubbers with at least one sorbing the carbon dioxide, and at least one other desorbing the sorbed carbon dioxide.

18. The method of claim 17, further comprising:
directing airflow towards the plural carbon dioxide scrubbers, wherein the plural carbon dioxide scrubbers are located in fixed positions in the HVAC system.

19. (canceled)

20. The method of claim 15, wherein the porous substrate is configured as a continuously moving loop.

21. The method of claim 6, wherein the removing step includes transferring the liquid carbon dioxide sorbent from a first position to a second position in a container, wherein the first position is prior to the liquid carbon dioxide sorbent contacting the circulated air, and wherein the second position is after the liquid carbon dioxide sorbent contacts the circulated air.

22. A heating, ventilation, and air-conditioning (HVAC) system comprising:
an air circulation system configured to circulate a first airstream within an enclosed environment;
an outdoor air inlet configured to introduce a second airstream in the enclosed environment;
a scrubber configured to remove carbon dioxide from the enclosed environment, via:
a carbon dioxide sorbent adhered, attached or entrapped within a porous substrate configured to move between a first position and a second position, or
a liquid carbon dioxide sorbent configured to move from a first position to a second position in a container; and
a means for desorbing the carbon dioxide from the sorbent.
